(19) United States
(12) Patent Application Publication
(10) Pub. No.: US 2017/0044586 A1
(43) Pub. Date: Feb. 16, 2017

(54) SOPHOROLIPID-CONTAINING COMPOSITIONS

(71) Applicant: Cargill, Incorporated, Wayzata, MN (US)

(72) Inventor: Anthony Louis DURAN, Pella, IA (US)

(21) Appl. No.: 15/305,732

(22) PCT Filed: Apr. 21, 2015
(86) PCT No.: PCT/US15/26799

§ 371 Date: Oct. 21, 2016

Related U.S. Application Data
(60) Provisional application No. 61/981,951, filed on Apr. 21, 2014.

(51) Int. Cl. C12P 19/44

(52) U.S. Cl. CPC C12P 19/44

(57) ABSTRACT

A process to produce a sophorolipid composition is disclosed, the steps including obtaining a sophorolipid containing composition having a pH of less than 5, adding 6 percent by weight or less of a free fatty acid to the composition, and thereafter adjusting the pH of the composition to a pH greater than 5. In some embodiments, the sophorolipid composition initially comprises from 4 to 80 percent by weight dry solids.

### BACKGROUND

Hydrocarbons are obtained from subterranean formations by drilling through a well that penetrates the formation. This provides a partial flow-path for the hydrocarbons to reach the surface. In order for the hydrocarbons to be produced, there must be a sufficiently unimpeded flow path from the formation to the wellbore to be pumped to the surface. Some wells need to be stimulated due to insufficient porosity or permeability of the formation. Common stimulation techniques include hydraulic fracturing and acidizing operations. The efficiency in hydrocarbon recovery from such stimulation techniques is dependent on the development of sufficient channels for the flow of hydrocarbons from low permeability regions of the formation.

During hydraulic fracturing, a fracturing fluid, typically a gelled or thickened aqueous solution containing proppant is injected into the wellbore under high pressure and high injection rates. Once natural reservoir pressures are exceeded, the fluid induces a fracture in the formation and transports the proppant into the fracture. The fracture generally continues to grow during pumping and the proppant remains in the fracture in the form of a permeable pack that serves to “prop” the fracture open. The fractures radiate outwardly from the wellbore and extend the surface area from which oil or gas drains into the well. The proppant pack forms a highly conductive pathway for hydrocarbons and/or other formation fluids to flow into the wellbore.

An efficient fracturing fluid should possess good proppant transport characteristics. Such characteristics are dependent on the viscosity of the fluid. Generally, the viscosity should be high in order to achieve wider and larger fractures. High viscosity is further generally desirable for more efficient transport of proppant into the fractured formation. The fracturing fluid therefore typically contains a viscosifying agent, such as a viscoelastic surfactant or a polymer. The polymer may be linear or cross-linked. In certain formations, aqueous acid solutions can be used to improve the permeability of the formation, thereby increasing hydrocarbon production. These acids are often combined with polymeric gels to provide an acid fracturing fluid.

A wide range of additives may be used to enhance the rheological properties and/or the chemical properties of the fluid. Such additives include viscosifiers, friction reducing agents, surface active agents and fluid loss control additives.

After the fracturing fluid is injected into the formation and fractures have been established, production of hydrocarbons is enhanced through the new fractures by removal of the viscous fluid. Generally, the viscosity of the fluid may be decreased by introducing breakers into the formation which degrade the polymer or break the emulsion. However, breakers often result in incomplete breaking of the fluid and/or premature breaking of the fluid before the fracturing process is complete.

Similar to stimulation fluids, other fluids used to treat wells must be removed following the completion of the treatment operation for which such fluids are used. For instance, polymeric viscosifying agents frequently used in drilling muds and well completion fluids have a damaging effect since they tend to interfere with other phases of drilling and/or completion operations, as well as production of the well after such operations are finished. For example, drilling fluids tend to seep into the surrounding formation forming a filter cake on the wall of the wellbore. The filter cake sometimes can prevent casing cement from properly bonding to the wall of the wellbore. It is important in such operations that the viscosifying agents and other components of the drilling mud be removed from the well in order to enhance the recovery of hydrocarbons. Oxidative breakers and enzymes are often used to degrade the polysaccharide-containing filter cakes and residual damaging materials which reduce their viscosity.

As an alternative to the use of breakers, or for use in conjunction with breakers, flowback additives are often introduced into the well to assist in the removal of well treatment fluids. Flowback additives are typically surfactants. Such surfactants reduce the surface tension between the treatment fluid, the formation, and/or hydrocarbons. For instance, in the recovery of hydrocarbon gases, flowback additives enable the recovery of more fluid which restores the formation's relative permeability to gas.

While conventional surfactants have been widely used as flowback additives for the removal of treatment fluids from the formation and well, such surfactants may not be environmentally friendly with respect to all relevant factors.

### SUMMARY OF THE INVENTION

One embodiment of the invention is an aqueous composition comprising about 4-80 percent by weight of dry solids comprising a mixture of ester-from sophorolipid and acidic-form sophorolipid, about 0.1-6 percent by weight formulated free fatty acid content, and less than 96 percent by weight water, wherein the aqueous composition has a pH greater than 5 and a flowback number in a 2% KCl of greater than 60. In some embodiments, the total solids of the inventive composition comprises from about 40 to about 99 percent by weight total sophorolipids based on the total solids. In other embodiments, the total solids of the invention composition comprises from about 70 to about 99 percent by weight, about 75 to about 95 percent by weight, or about 80 to about 99 percent by weight total sophorolipids based on the total solids. In still other embodiments, the total solids of the inventive composition comprises at least 60 percent by weight, at least 70 percent by weight, or at least 75 percent by weight total sophorolipids based on the total solids.

In some embodiments, this composition is suitable for use as a flowback additive in a natural gas or crude oil fraccing application (also called fracking application).

In some embodiments, these sophorolipid-containing compositions, when measured using the flowback test set out in Example 5 below, provide a measured flowback number of at least 60, at least 65, at least 70, at least 75, at least 77, at least 80, or at least 85 in a 2% KCl Solution (as described in the examples). In other embodiments, these sophorolipid-containing compositions, when measured using the flowback test set out in Example 5 below, provide a measured flowback number of at least 60, at least 65, at least 70, at least 75, at least 77, at least 80 or at least 85 in Hard Water (as described in the examples).

In other embodiments, the sophorolipid containing compositions may be used with a pour point depressant to provide desired pour properties or pourability. Suitable pour point depressants for use with this invention include, but are not limited to, glycerol, propanol, ethanol, methanol, butanol, polyethylene glycol, polypropylene glycol, ethylene glycol, propylene glycol, or mixtures thereof.

Another embodiment of the invention is a process to produce a sophorolipid composition, the process comprising: (a) obtaining a sophorolipid containing composition comprising about 4-80 percent by weight dry solid, comprising at least one sophorolipid, wherein the sophorolipid containing solution exhibits a pH of less than 5, (b) adding about 6 percent by weight or less of a least one fatty acid to the sophorolipid containing composition to provide a fatty acid adjusted sophorolipid composition, and (c) adjusting the pH of the fatty acid adjusted sophorolipid composition to a pH greater than 5 to provide the sophorolipid composition. In some embodiments, the total dry solids of the sophorolipid composition comprises from about 40 to about 99 percent by weight total sophorolipids based on the total dry solids. In other embodiments, the total dry solids of the sophorolipid composition comprises from about 70 to about 99 percent by weight, about 75 to about 95 percent by weight, or about 80 to about 99 percent by weight total sophorolipids based on the total dry solids. In still other embodiments, the total dry solids of the sophorolipid composition comprises at least 60 percent by weight, at least 70 percent by weight, or at least 75 percent by weight total sophorolipids based on the total dry solids.

In some embodiments, the composition produced by this process is suitable for use as a flowback additive in a natural gas or crude oil fraccing application (also called fracking application).

in an embodiment, the sophorolipids may be a mixture of acidic-form sophorolipids of formula (Ia), where the sophorolipids may be in the free acid form (—R³—COOH); or acidic-form sophorolipids of formula (Ib), where the acidic-form sophorolipids may be in the neutralized form, as a salt or as a sophorolipid anion (as illustrated in formula (Ib) below) and associated cations (i.e. NH₄⁺, Na⁺, Ca²⁺, Mn²⁺, or Fe³⁺, typically Na⁺ or K⁺) that are distributed in the sophorolipid containing composition and n is 1, 2, or 3.

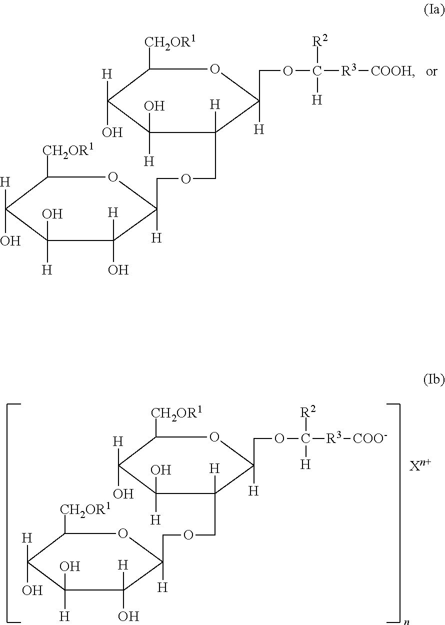

and ester-form sophorolipids of formulas either (IIa) or (IIb), or mixtures of (IIa) and (IIb), where these ester-form sophorolipids may be in the closed-ring form (lactone) that may also be referred to as lactonic sophorolipids, or where the sophorolipids are in the open-ring form but the carboxyl acid moiety is esterified with, for example, a suitable alcohol or other hydroxyl-containing compound (—R³—COOR⁴, as an ester),

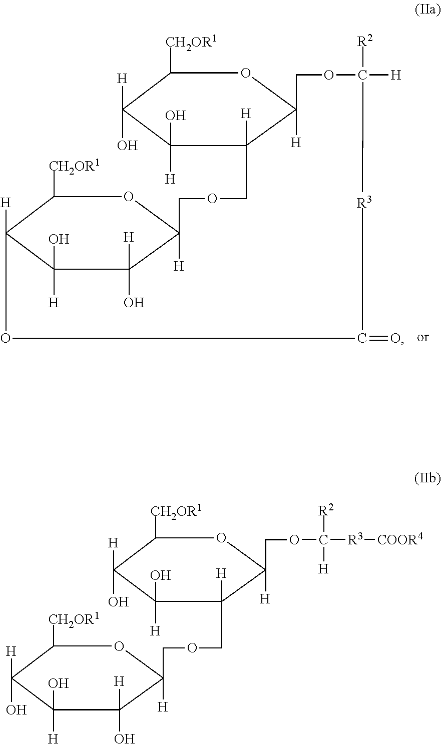

wherein R¹ is hydrogen, a C₁ to C₄ hydrocarbon or carboxylic acid group (typically an acetyl group); and either (i) R² is hydrogen or a C₁-C₉ saturated or unsaturated aliphatic group; and R³ is a C₇-C₂₀ saturated or unsaturated aliphatic group; or (ii) R² is hydrogen or a methyl group and R³ is a saturated or unsaturated hydrocarbon chain that contains from 7 to 20 carbon atoms. Typically R² is a hydrogen or methyl or ethyl group, (preferably a methyl group or hydrogen). Typically R³ is C₇ to C₂₀ saturated or unsaturated aliphatic group a C₇ to C₂₀ (preferred is C₁₅ monounsaturated), and R⁴ is hydrogen, C₁-C₉ saturated or unsaturated aliphatic group, monohydroxyl aliphatic group, or polyhydroxyl aliphatic group (preferred is hydrogen group).

In one embodiment, the sophorolipid is a mixture of sophorolipid compounds of the formulas (Ia) and (IIa) wherein R² is hydrogen or a C₁ to C₄ hydrocarbon (typically methyl).

In another embodiment, the sophorolipid is a mixture of acidic-form sophorolipids where at least portion of the acid moiety is neutralized with a base to form a salt or where the sophorolipid anion and associated cations of formula (Ib), as described above, are distributed in the sophorolipid containing composition, and ester-form sophorolipids as described in formulas (IIa) and (IIb). In yet another embodiment, all or any combination of the forms of the above describe sophorolipids may be in the composition.

In some embodiments, the process includes adding a biocide to the fatty acid adjusted sophorolipid composition. In other embodiments, the aqueous sophorolipid composition includes a biocide. Suitable biocides include those known to one of ordinary skill in the art. Preferably, the biocides can be utilized at levels that are nontoxic but that provide effective antimicrobial activity. In some embodiments, suitable amounts of effective biocides are about at least 10 ppm (0.001 percent by weight), at least 50 ppm (0.005 percent by weight), or at least 100 ppm (0.01 percent by weight). In other embodiments, suitable amounts of effective biocides are about less than 1 percent by weight (10000 ppm), less than 0.5 percent by weight (5000), or less than 0.05 percent by weight (500 ppm). Suitable ranges of effective biocides are 100-500 ppm (0.01-0.05 percent by weight), 100-5000 ppm (0.01-05 percent by weight) or 100-10000 ppm (0.01-1 percent by weight).

### DETAILED DESCRIPTION

### Sophorolipids

Sophorolipids may be manufactured from a lipid source with variations in the process being dependent on the organism being utilized, the equipment and fermentation protocol, and the production medium utilized.

Typically the organism utilized is a yeast strain, preferably a non-pathogenic yeast strain. The lipid source can be oils derived from plant-based oils and/or animal sources, animal fats, or free fatty acids derived from one or more of these sources. Typically, the oleic acid content of the lipid source contains at least 40 percent by weight oleic acid (or for monoacyl glycerides, diacyl glycerides, and triacyl glycerides the oleic acid component is esterified to a glycerol residue). When higher flowback numbers are desirable the oleic acid content of the lipid source typically is at least 50 percent by weight, preferably at least 60 percent by weigh of the total fatty acid content. For a particularly preferred embodiment, the oleic acid content is at least 70 percent by weight of the total fatty acid content, and for ease of manufacturing, preferably a mixture of free fatty acids is utilized, wherein the oleic acid content of the free fatty acids is at least 60 percent by weight, preferably at least 70 percent by weight. For this application, “fatty acid content” includes both free fatty acids and fatty acids derivatives that are esterified to glycerol or another alcohol, and oleic acid content includes both free oleic acid and derivatives of oleic acid that are esterified to glycerol or another alcohol. It is believed that higher oleic acid content will result in a higher ratio of ester-form sophorolipid to acidic-form sophorolipid. The lipid source may comprise fatty acid distillates derived from plant-based oils, animal fats, or fish oils having fatty acid content as described above. For purposes of this application, fatty acid distillates are mixtures of free fatty acids.

Sophorolipids are naturally occurring bio-surfactant glycolipids produced from yeasts. For instance, the sophorolipids are glycolipids produced fermentatively from such yeasts as Candida bombicola, Starmerella bombicola, Candida apicola, Candida tropicalis, Candida gropengiesseri, Candida batistae, Candidafloricola, Starmerella floricola, Candida riodocensis, Starmerella riodocensis, Candida riodocensis, Candida stellate, Starmerella stellata, Candida sp. NRRL Y-27208, Rhodotorula bogoriensis, Pichia anomala, Trichosporon asahil and Wickerhamiella domercgigge Candida bombicola, Candida qpicola, and Wickerhamiella domercgiac. Sophorolipids are generally composed of a dimeric sophorose sugar moiety (β-D-Glc-(1→2)-D-Gle) linked glycosidically to a hydroxyl fatty acid residue. In a preferred embodiment, a sophorose sugar moiety is linked via the glycosidic linkage to the hydroxyl group of a 17-hydroxy-C₁₈ saturated or monoenoic (cis-9) fatty acid.

Depending on the pH of the system, the acidic-form sophorolipids will be in a linear, free acidic sophorolipid form, or a linear, neutralized acidic sophorolipid form. To a lesser extent, the pH of the system may also impact the degree to which the sophorolipids assume a closed ring esterified or lactonic sophorolipid form, or a linear, esterified sophorolipid form. In addition, the 6-hydroxyl groups of the glucose moieties may be acetylated or free hydroxyl groups. Depending upon the organism and the fermentation conditions used (including, but not limited to, the lipid source utilized) to produce the sophorolipid, the acidic-form or esterified-form may predominate.

As described above, the sophorolipids may be a mixture of acidic-form sophorolipids of formula (la), where the sophorolipids may be in the free acid form (—R³—COOH); or acidic-form sophorolipids of formula (Ib), where the acidic-form sophorolipids may be in the neutralized form, as a salt or as a sophorolipid anion (as illustrated in formula (Ib) below) and associated cations (i.e. NH₄⁺, Na⁺, K⁺ Ca²⁺, Mn²⁺, or Fe³⁺, typically Na⁺ or K⁺) that are distributed in the sophorolipid containing composition and n is 1, 2, or 3.

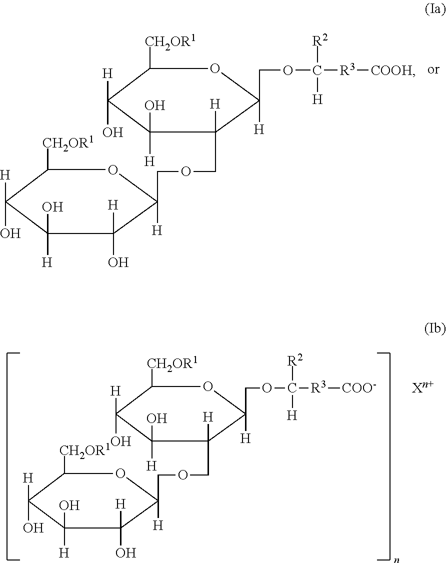

and ester-form sophorolipids of formulas either (IIa) or (IIb), or mixtures of (IIa) and (IIb), where these ester-form sophorolipids may be in the closed-ring form (lactone) that may also be referred to as ester sophorolipids, or where the sophorolipids are in the open-ring form but the carboxyl acid moiety is esterified with, for example, a suitable alcohol or other hydroxyl-containing compound (—R³—COOR⁴, as an ester),

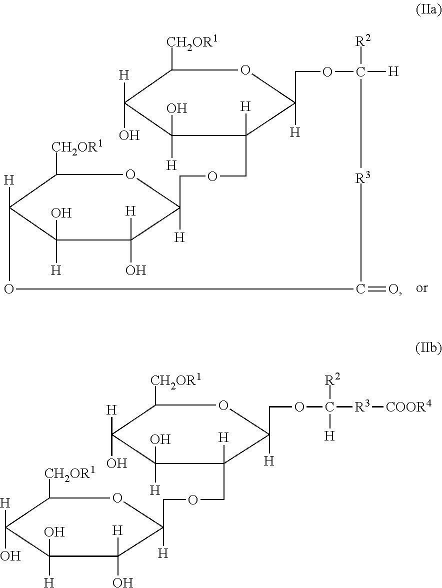

wherein R¹ is hydrogen, a C₁ to C₄ hydrocarbon or carboxylic acid group (typically an acetyl group); and either (i) R² is hydrogen or a C₁-C₉ saturated or unsaturated aliphatic group; and R³ is a C₇-C₂₀ saturated or unsaturated aliphatic group; or (ii) R² is hydrogen or a methyl group and R³ is a saturated or unsaturated hydrocarbon chain that contains from 7 to 20 carbon atoms. Typically R² is a hydrogen or methyl or ethyl group, (preferably a methyl group or hydrogen). Typically R³ is C₇ to C₂₀ saturated or unsaturated aliphatic group a C₇ to C₂₀ (preferred is C₁₅ monounsaturated), and R⁴ is hydrogen, C₁-C₉ saturated or unsaturated aliphatic group, monohydroxyl aliphatic group, or polyhydroxyl aliphatic group (preferred is hydrogen group). In one embodiment, the sophorolipid is a mixture of sophorolipids compounds of the formulas (Ia), (Ib), (IIa), and/or (IIb) wherein R² is hydrogen or methyl.

In another embodiment, the sophorolipid is a mixture of acidic-form sophorolipids where the acid moiety is at least partially neutralized with a base to form a salt or anion and cation distributed in the sophorolipid containing composition as described above, and ester-form sophorolipids where the carboxylic moiety is a lactone or an open chain ester-form sophorolipid (i.e. where the lactone ring is in open form but the acid moiety is esterified with a suitable hydroxyl containing compound such as, for example, glycerol or some other hydroxyl containing compound, such as mono- and poly-alcohols), or mixtures thereof. In yet another embodiment, all or any combination of the above describe sophorolipids may be in the composition.

A representative fermentation method to prepare suitable sophorolipids is set out in Example 1, described below.

Fermentations proceed by addition of carbon source, typically in the form of sugar, fatty acid source in the form of oil or partially distilled and purified free fatty acids, water and nutrients necessary for cell propagation such as salts, nitrogen source, etc. into a temperature controlled vessel with airflow provided to oxygenate the broth. This fermentation can be fed additional carbon source or lipid source during fermentation. The order of nutrient, carbon source, and lipid source addition can be varied based on the fermentation process and equipment utilized, as known by one of skill in the art in light of the teachings contained herein. Fermentations are provided with enough airflow to maintain at least a partially aerobic environment throughout fermentation.

Fermentation conditions are selected, for example, to provide a ratio of ester-form to acidic form sophorolipids of about at least 1:1, at least 6:4, at least 7:3, or at least 8:1 when measured using the analytical method set out in Example 4, described below. Typically, the ratio of ester-form to acidic-form sophorolipids in the composition is less than 99:1, typically less than 95:1, for example less than 9:1.

In addition to being biodegradable, the sophorolipid biosurfactants are non-toxic, biocompatible and are made from renewable resources. The use of sophorolipid biosurfactants in well treatment fluids provides a green alternative to treatment fluids containing conventional flowback surfactants. In well treatment operations, such as hydraulic fracturing, sophorolipid biosurfactants provide an attractive alternative to conventional synthetic surfactants. They further maximize the benefits of a fracturing operation by improving the recovery of the treatment fluid introduced into the formation. Fermentation broth containing the sophorolipid typically is agitated while heating to the desired settling temperature. The agitation and heating enhances the migration of the sophorolipid into an organic-enriched phase that can be readily separated from an aqueous-enriched phase. Agitation typically is discontinued when the desired temperature is reached. Agitation and heating of the fermentation broth enhances the separation of the broth into an aqueous-enriched phase and an organic-enriched phase. The heated agitated fermentation broth is allowed to gravity settle until the desirable separation of the aqueous-enriched phase and organic-enriched phase has been obtained. The organic-enriched phase containing crude sophorolipids typically is collected from the bottom of the vessel while the aqueous-enriched phase typically is left in the fermentation vessel. If desired, the fermentation broth can be moved to another vessel before the crude sophorolipid product is recovered. This allows another fermentation to be carried out while the crude sophorolipid product is being recovered from the previous fermentation broth. In some aspects this second vessel contains both heating and agitation apparatus for enhancing the recovery of the crude sophorolipid.

### Pour Point Depressant

The pour point depressant is a compound containing at least one hydroxyl group that improves the low temperature properties of the sophorolipid-containing composition. Suitable pour point depressants include glycerol, propanol, ethanol, methanol, butanol, polyethylene glycol, polypropylene glycol, ethylene glycol, propylene glycol, or mixtures thereof. In one embodiment the pour point depressant includes glycerol, ethanol, methanol, propanol, ethylene glycol, propylene glycol, or mixtures thereof. In other embodiments the pour point depressant includes glycerol, USP glycerol, crude glycerol, low sodium crude glycerol (i.e. less than 0.3% ash), tech-grade glycerol, glycerol fleeting USP glycerin specifications, or mixtures thereof. A suitable pour point depressant exhibits or provides a sophorolipid composition having pourability as defined in the analytical method of 30° F. or lower, a pourability of 10° F. or lower, a pourability of 0° F. or lower, a pourability of −10° F. or lower, or a pourability of −20° F. or lower when measured after 24 hours using the pourability method set out below. Typically, the sophorolipid composition will remain flowable using the pourability test for at least 1, 2, 3, 4, 5, 6, 7, 8, 9, 10, 11, 12, and/or 13 weeks. Another method for determining the low temperature properties of the sophorolipid composition is by determining the Pour Point for the composition according to ASTM D97 standard test method for pour point of petroleum products. ASTM Standard D97-07, 2007, “Standard Test Method for Pour Point of Petroleum Products,” ASTM International, West Conshohocken, Pa., 2007, DOI: 10.1520/D0097-07. www.astm.org.

The pourability of sophorolipid composition may be determined as described in the analytical methods using the conditions described in the examples. The temperature being tested and the time period being evaluated are varied as needed to evaluate the composition.

### Sophorolipid-Containing Formulation

The sophorolipids that are recovered from the organic enriched layer of the fermentation broth are used to prepare compositions of the present invention.

Sophorolipids separated from fermentation broth (as described above) typically are combined with water, if desired. While agitating, the sophorolipid containing material is neutralized with a caustic solution to reach a desired pH (typically to a pH value at least about 5, at least about 6, or at least about 7. Typically, pH values are 12.5 or less, 11 or less, or in a range of about 6-8.5.

The dry basis solids of the sophorolipid containing formulation is adjusted so that the formulation will have from about 4-80 percent by weight total solids of the composition. The dry basis total solids are adjusted using conventional techniques by adding or evaporating the amount of desired water to the formulation. In some embodiments, the total solids of the formulation is about 16-31 percent by weight total solids (excluding the pour point depressant), in other embodiments the total solids is about 4-50 percent by weight total solids (excluding pour point depressant).

The weight percent of the total solids of the inventive composition (excluding any pour point depressant) will contain about 40-90 percent by weight total sophorolipids. In other embodiments, the total solids of the invention composition (excluding any pour point depressant) comprises about 70 to about 99 percent by weight, about 75 to about 95 percent by weight, or about 80 to about 99 percent by weight total sophorolipids based on the total solids. In still other embodiments, the total solids of the inventive composition (excluding any pour point depressant) comprises at least 60 percent by weight, at least 70 percent by weight, or at least 70 percent by weight total sophorolipids based on the total solids. The pH may be adjusted with an aqueous base such as, for example, a NaOH aqueous solution. Sufficient aqueous base is added so that the pH of the final composition will exhibit a pH of typically 12.5 or less, a pH of 12 or less, a pH of 11 or less, a pH from about 7-11, or a pH of from about 6-8.5. While the resulting formulation(s) may be used for numerous end-use applications, the formulations are particularly suitable for use as a flowback additive in a natural gas or crude oil fraccing application and as additive for use in workovers of natural gas or crude oil wells, including, but not limited to, acidization workovers.

In an embodiment, free fatty acid is added to the sophorolipid-containing organic enriched layer before the addition of base to adjust the pH. The free fatty acid typically is selected to have high oleic acid content and is added in an amount sufficient to provide a free fatty acid content of about 0.1-6 percent by weight of the composition, about 0.5-5 percent by weight of the composition, about 0.5-2.5 percent by weight of the composition, or about 0.5-2 percent by weight of the composition and in some instances about 0.5 to 1.5 percent by weight of the composition (sometimes referred to as the formulated free fatty acid content). For purposes of this application, the free fatty acid content in the sophorolipid containing compositions described herein includes free fatty acid in the of form of free fatty acids, the portion of a neutralized fatty acid salt attributable to the fatty acid moiety, and anions of free fatty acids distributed in an aqueous sophorolipid containing compositions, and/or mixtures of these forms of free fatty acid. Preferably, the free fatty acids utilized are fatty acid distillates.

The final sophorolipid composition, when measured using the flowback test set out in Example 5 below, provide a measured flowback number of at least 60, at least 65, at least 70, at least 75, at least 77, at least 80, or at least 85 in a 2% KCl Solution (as described in the examples). In some aspects the final composition, when measured using the flowback test set out in Example 5 below, provides a measured flowback number of at least 60, at least 70, at least 75, at least 77, at least 80 or at least 85 in Hard Water (as described in the examples).

### Biocide

In an embodiment, the sophorolipid-containing compositions include a biocide. The biocide may be added to the composition during the process described herein using methods known to those skilled in the art. Suitable biocides include materials or mixtures of materials that are microbial effective and stable in compositions having a pH greater than 5, 6, 7, 8, 9, 10, 11 or 12. In some embodiments, suitable amounts of effective biocides are about at least 10 ppm (0.001 percent by weight), at least 50 ppm (0.005 percent by weight), or at least 100 ppm (0.01 percent by weight). In other embodiments, suitable amounts of effective biocides are about less than 1 percent by weight (10000 ppm), less than 0.5 percent by weight (5000), or less than 0.05 percent by weight (500 ppm). Suitable ranges of effective biocides are 100-500 ppm 0.01-0.05 percent by weight), 100-5000 ppm (0.01-05 percent by weight) or 10040000 ppm (0.01-1 percent by weight).

### EXAMPLES

### Analytical Methods and Materials Utilized

Pourability is determined by adding sample solution to a 50 mL centrifuge tube and placing it into a freezer at the appropriate temperature (for example, if the pourability at −20° C. is being determined the freezer is set at −20° C./−4° F.). After twenty four hours in the freezer, the tubes are tilted to check for pourability. If the material moves in the tube then the sample result is reported as “flows”. If the material does not move then the sample is considered “frozen”. The same method is used to determine the pourability of the samples at longer periods of time, with the sample being indicated to flow or not after the desired test period.

The ratio of ester-form sophorolipids to acidic-form sophorolipids in a formulated composition is determined according to the method described in Example 4, below,

Fatty Acid Distillate: is a mixture of free fatty acids derived from animal sources and typically comprises 73% oleic acid, 8% linoleic acid, 6% paimitoleic acid, and 1% linolenic acid (CAS# 112-80-1); available from Bremitag Great Lakes under the product name Emersol 213 NF.

95 Dextose: A concentrated dextrose with a minimum dextrose concentration of 94% dextrose and a pH of 5 with a dry solid content of 70.5-71.5 percent by weight available from Cargill, Incorporated.

OHLY-KAT: Yeast extract, available from OHLY Americas.

Solulys 095E: Spray-dried corn steep with 24 wt % lactic acid, 44 wt % protein, 18 wt % ash, 1 wt % sugars, 13% other elements, available from Roquette Chemicals & Bio-Industries.

Magnesium Sulfate Heptahydrate: available from J.T. Baker under the product designation 2505-07, VWR.

Ammonium Phosphate Dibasic: available from J.T. Baker under the product designation 0784-07, VWR.

Ammonium Sulfate: available from J.T. Baker under the product designation 0792-07, VWR.

Ferrous Sulfate Heptahydrate: available from Fisher Scientific under the product designation 1146-500.

Manganous Sulfate Monohydrate: available from Midland Scientific under the product designation 2550-01, J.T. Baker.

Zinc Sulfate Hep ydrate: available from Fisher Scientific under the product designation Z76-500.

USP Glycerol: 99.0 wt % glycerol available from Baker Baker under the product designation 4043-00.

Crude Glycerol: 84% Glycerol, 11% Water, 2% NaCl, 1% Methanol, 2% Organic Residue, available from Cargill, Incorporated.

Ultra-Pure Water; 18 megohm resistivity water made using a water purification system available from Hydro Service and Supplies.

Tap Water: Cl 6.5 ppm; Cu 0.117 ppm; K 0.01 ppm; Mg 0.002 ppm; Mn 0.279 ppb; Na 0.998 ppm; P 0.014 ppm; and Zn 0.21 ppm, in aqueous solution available from the Rathbun Regional Water Association.

Hard Water: an aqueous solution containing CaCl₂-2H₂O 1.03 wt %; MgCl₂-6H₂O 0.56 wt %; and NaCl 3.76 wt %.

2% KCl Solution: an aqueous solution is prepared by accurately weighing 20g KCl and adding it to 980 g of Tap Water. KCl available from Midland Scientific under the product designation 3040-05, J.T. Baker.

All the percentages listed are weight percentages (wt %), unless otherwise indicated to the contrary.

### Example 1

### Sophorolipids from Fermentation

Starmerella bombicola NRRL Y-17069 was obtained from the Agricultural Resource Service (ARS) Culture Collection. The original culture is plated for purity on a potato dextrose agar (PDA) plate. A single colony is selected from the plate and used to inoculate a 250 ml shake flask containing 50 ml of sterilized Yeast Mold (YM) broth. The shake flask is placed in a shaker incubator overnight (25° C. and 250 rpm). Following overnight incubation, sterile 80% glycerol is added to the seed broth to make a glycerol seed stock at a final glycerol concentration of 20%. One ml aliquots are added to cryo-vials and stored in a −80° C. freezer.

Pre-cultures are prepared by inoculating a 250 ml shake flask containing 50 ml of autoclaved YM broth with a single cryo-vial (1 ml glycerol stock) and incubating it in a shaking incubator (25° C. and 250 rpm) for 24 hours. The 50 ml culture is used to inoculate a 14 L New Brunswick fermenter containing 10 L of autoclaved Sophorolipid (SL) Seed medium at an OD600 of 0.02. The SL seed medium consists of 30 g/L dextrose, 48 g/L OHLY-KAT yeast extract and trace minerals (10 mg/L ferrous sulfate (heptahydrate), 2 mg/L manganous sulfate (monohydrate), 15 mg/L zinc sulfate (heptahydrate). The seed fermentor temperature is controlled at 25° C. Agitation begins at 550 rpm and is cascaded to a maximum of 1100 rpm to maintain a minimum % dissolved oxygen of 40 throughout the fermentation. The pH is not maintained. The seed culture is harvested at or near the peak oxygen uptake rate (OUR) (typically 115-130 at 29-30 hours).

The main fermentation Sophorolipid medium consists of 4 g/L dry basis nitrogen source (either raw light steep water or Solulys 095E), 1.65 g/L ammonium sulfate, 1.06 g/L ammonium phosphate (dibasic), 0.5 g/L magnesium sulfate (heptahydrate) and 2 mg/L thiamine-HCl. The starting dextrose concentration is 100 g/L (+/−20) and the starting lipid source concentration is 30 g/L (+/−10).

Main fertnentors are inoculated to an OD600 of 2.8 with S. bombicola 10 L seed culture. Salts, dextrose feeds and oil feeds are sterilized separately. The initial pH of the media is approximately 5.2 and is allowed to naturally drop and is maintained at 3.5 for the remainder of the fermentation with 2N NaOH. The fermentation temperature is maintained at 30° C., aeration is set at one volume of air per volume of medium per minute (VVM) based on initial volume. Agitation is maintained at a level that allows for a peak oxygen uptake rate (OUR) of 50 (+/−5) mmol 1⁻¹ h⁻¹ following exponential cell growth and slowly trends down as the fermentation progresses due to increased fermentor volume and gradual slowing of cellular metabolism within an OUR range of 31 (+/−5) mmol 1⁻¹ h¹.

For the feed media, two addition vessels are utilized. One contains the sterilized lipid source and the other contains sterilized ˜600 g/L 95 Dextrose. 95 Dextrose is fed into the fermentor to maintain a fermentation broth concentration of 25 g/L (+/−20) after an initial drop from the starting concentration of 100 g/L (+/−20). The lipid source is fed into the fermenter between 9 and 40 hours of elapsed fermentation time. A total of 200 g/L of the lipid source is added (based on starting fermentation volume). As the lipid source is nearing depletion the dextrose feed is reduced or stopped to allow for both levels to reach near 0 g/L at the end of fermentation (EOF). EOF is determined by neutral lipid depletion (based on hexane extraction) and a free fatty acid content of <2.5 g/L (as measured by high pressure liquid chromatography with an evaporative light scattering detector (HPLC/ELSD). The final dextrose concentrations should be 5 g/L or less.

### Example 2

### Method for Obtaining a Fraction Enriched in Sophorolipids

Heat treatment of the sophorolipid fermentation broth is conducted by heating the vessel to 70° to 75° C. along with minimal agitation to facilitate an adequate heat transfer. Once the fermentation broth reaches 70° to 75° C., the agitation is stopped. The fermentation broth is then allowed to naturally cool as the sophorolipid product layer physically separates from the aqueous layer within the fermentation broth due to differences in density. The broth is allowed to gravity settle for a minimum of 30 minutes. The organic phase containing enriched crude sophorolipids is collected from the bottom of the vessel and the aqueous phase is left in the vessel.

The ratio of ester-form sophorolipids to acidic-form sophorolipids for various crude sophorolipid fermentation samples that have been prepared by the process related to this example is provided in Table 1. Samples 2-1 through 2-6 exhibit a formulated free fatty acid content of from 0.14 to 1.6 percent by weight of the sample.

TABLE 1
Ratio of Ester-From to Acidic-Form Sophorolipids

| Crude Sophorolipid | Fermentation | Ratio of Ester-form sophorolipid |
|---|---|---|
| Sample (No.) | Sample (No.) | to Acidic-form sophorolipid |
| 2-1 | 1-1 | 1.50 |
| 2-2 | 1-2 | 1.61 |
| 2-3 | 1-3 | 1.23 |
| 2-4 | 1-4 | 1.15 |
| 2-5 | 1-5 | 1.08 |
| 2-6 | 1-6 | Not Analyzed |

### Example 3

### Formulation with Pour Point Depressant

Crude sophorolipid (containing 48-57% dry solids, 50% water and exhibiting a pH of 15 to 3.8) measures of 464 g (230 g dry weight) of samples similar to 2-1 to 2-6 are each mixed with 14 g Ultra-Pure water. Each of the resulting solutions is neutralized using 10 g of 50% sodium hydroxide to achieve a pH of 6.9-7.1. USP grade glycerol, 522 g (520 g dry weight), is added to each of the solutions. The formulated solutions contain 23% dry weight as crude sophorolipid, 52% dry weight as glycerol and 25% as water. The characteristics of the formulated sophorolipid are detailed in Table 2.

TABLE 2
Characteristics of Formulated Pour Point Depressant Samples

| | Crude Sophorolipid Sample (No.) | | | | |
|---|---|---|---|---|---|
| | 2-1 | 2-2 | 2-3 | 2-4 | 2-5 |
| | Formulated Sample(No.) | | | | |
| | 3-1 | 3-2 | 3-3 | 3-4 | 3-5 |
| Density (g/mL) | 1.18 | 1.19 | 1.19 | 1.19 | 1.19 |
| Refractive Index | — | 1.46 | 1.45 | 1.44 | 1.44 |
| Conductivity (μS) | — | 485 | 504 | 428 | 426 |
| Viscosity at 30° C. | — | 154 | 123 | 171 | 183 |
| pH | 7.12 | 7.07 | 7.01 | 7.07 | 7.12 |
| CMC (mg/L) | 214 | 308 | 370 | 353 | 320 |

### Example 4

### Method to Determine Ratio of Esterform Sophorolipids to Acidic-Form Sophorolipids

The ratio of ester-form to acidic-form sophorolipids is determined on representative samples from Example 3 using an LCMS-based method. Samples are diluted in 50% acetonitrile and analyzed using a Dionex Summit HPLC System equipped with a Waters XBridge C18, 5 μm, 2.1 ID×150 mm column at a flow rate of 0.4 mL/min using a gradient shown in Table 3. Mass is detected using a Thermo Exactive mass spectrometer with a negative scan mode, scan range of 150-2000 mass-to-charge ratio (m/z), scan time of 30 min, electrospray ionization mode with a spray voltage of 4.0 kV, and a capillary temperature of 200° C. The mass spectrum is then filtered to only display masses of 500-750 m/z, which is the typical mass range for sophorolipids. The acidic-form fraction is defined as all peaks eluting between around 10-14 minutes with the first peak having a ink of 595 and last peak having a m/z of 707. Likewise, the ester-form fraction is defined as all peaks eluting between around 18-26 minutes with the first peak having a m/z of 603 and the last peak having a m/z of 689 (Chart 1). The peaks for the other ester-form fractions can be determined by utilizing the appropriate peaks in the LC method by procedures known to one of skill in the art. The ratio of ester-form to acidic form sophorolipid is defined as the ratio between the total area under the ester-form fraction peaks to the total area under the acidic-form fraction peaks on the chromatogram.

TABLE 3
LCMS gradient profile.

| Time (min) | Solvent A (%) | Solvent B (%) |
|---|---|---|
| 0 | 5 | 95 |
| 20 | 70 | 30 |
| 23 | 70 | 30 |
| 25 | 5 | 95 |
| 30 | 5 | 95 |

Solvent A = Acetonitrile (LCMS grade) with 0.1% NH₄OH,
Solvent B = Water (LCMS grade) with 0.1% NH₄OH

Chart 1: An exemplary MS spectrum when filtered from m/z 500-750 is set out below illustrating the acidic-form fraction and the ester-form fraction.

For the examples set forth in this application, the ratio of ester-form to acidic-form is an approximation based on the ratio of lactonic sophorolipid to acidic-form sophorolipid determined by using the method set forth above regarding identifying the ratio of lactonic sophorolipid and acidic-form sophorolipid. It is believed that any additional ester-form. sophorolipids present could be readily identified by appropriate determination of the peaks associated with such additional ester-form sophorolipids. It is believed that for the examples set forth below, the amount of additional ester-form sophorolipids present is small and therefore would only slightly increase the ratio of ester-form sophorolipids to acidic-form sophorolipids from the values listed.

### Example 5

### Determining Flowback Number

### Sample Preparation

The following flowback method is used to determine the flowback numbers associated with the sophorolipid formulations of this invention. For flowback number determinations, aqueous solutions containing the neutralized sophorolipid samples are prepared at 0.1 percent by weight in 2% KCl Solution or 0.1 percent by weight in Hard Water. The same method can be used for measuring flowback numbers in other aqueous solutions such as Ultra-Pure Water. The resulting aqueous solution(s) are tested to determine the flowback numbers in accordance with the method described below. The flowback numbers are reported together with the aqueous solution that was utilized to conduct the test.

### Apparatus

The flowback column consists of a clear polyacrylic column (8-inch length and 1-inch inner diameter), a Teflon bottom cap with 2 O-rings and 1 screen and a Teflon top cap with 2 O-rings and 1 screen. The top Teflon cap is differentiated from the bottom by a small hole drilled into the top of the cap. An outlet tube is attached to the top of the column. A 3-way valve is attached to the bottom of the column to control nitrogen flow to the column.

### Packing the Column and Loading a Sample

An 80-100 g neutralized sophorolipid sample (Flowback fluid) is prepared as describe above and 190 g Unimin. Unifrae 20/40 white sand is weighed out. The bottom column cap with a capped compression fitting is screwed on to the column. About 35 g flowback fluid is slowly added into the column through the end of the column. The Unifrac sand is slowly added into the column under mild vortexing (about 1300 rpm). When the level of the sand is just below the level of the fluid, more flowback fluid is added in 0.5 to 3 mL increments by syringe. The addition of sand and flowback fluid is continued until the level of sand is just above the top of the column. Once the column is filled, an O-ring is placed on top of the column followed by a screen and the second O-ring on top of the screen. Then the column top cap containing a compression fitting and a hole for air bubble elimination that will fit the tip of a 1 ml syringe is screwed on to the column. The flowback fluid is added through the top compression fitting until the liquid level is just below the top of the syringe hole. The column is placed on the vortex (about 1300 rpm) to remove any air bubbles. Additional fluid is added until the fluid level in the syringe hole no longer drops. The hole is then plugged with a 1 mL syringe or suitable plug. Additional flowback fluid is added via syringe to the top of the column such that the fluid level is over the lip of the compression fitting. The column is placed on the vortex to remove any remaining air bubbles. If the liquid level no longer drops, place the syringe needle on the lip of the compression fitting and remove excess liquid. A compression fitting cap is placed on the top of the column.

The 3-way valve with the flow switched away from the column is attached to the bottom of the column. The outlet tube is attached to the top of the column. The remaining sand and flowback fluid is weighed.

### Calibration of Nitrogen Flow

The nitrogen flow to the flowback column must he calibrated before each analysis. Tap Water (1000 mL) is added to a 1 L filter flask equipped with a rubber stopper that is fitted with a piece of plastic tubing. The tubing is inserted into a 1000 mL graduated cylinder above the 700 mL mark. The same nitrogen line used for flowback determination is connected to the filter flask and the nitrogen flow is turned on to the flask. The time it takes to displace 550 mL Tap Water out of the filter flask and into the graduated cylinder is measured using a stopwatch. The measured time period begins when the level reaches the 100 mL mark and ends at the 650 ml. mark. Nitrogen flow is adjusted until it takes between 49.5 and 50.5 seconds to collect 550 mL of Tap Water which corresponds to a nitrogen flow of 11 mL/sec.

Nitrogen flow rate is calculated as follows:

$$Flow Rate = \frac{550 ml}{Time}$$

### Flowback Data Collection

After the calibration of nitrogen flow the plastic tube attached to the column outlet is inserted into an empty graduated cylinder and is placed on a balance and tared. The three way valve is turned on to the column so that the gas flow is now passing through the column. The flowback start time is recorded. The weight of the fluid recovered is shown on the balance and fluid is recovered until the weight increase is less than 0.4 g per 10 minutes. The three way valve is then turned off and the weight of graduated cylinder with the recovered fluid is recorded.

The flowback number is calculated as follows:

$$Flowback Number = \frac{Weight of recovered solution}{Starting weight of flowback solution in the column} * 100$$

Hard Water is tested to ensure proper column standardization. The expected flowback number for Hard Water is 54.8 +/−3 (i.e. 51.8-57.8 respectively). The column and/or operation of the column should be adjusted if the flowback number for Hard Water is outside the 51.8-57.8 range.

### Example 6

### Testing Flowback Samples with Pour Point Depressant and No added Free Fatty Acid

Crude sophorolipids of samples 2-2, 2-3, 2-4, 2-5 and 2-6 are mixed with the glycerol as indicated in Table 5 and Ultra-Pure Water in a sample tube. The sample tube is placed on a shaker for 5 minutes and the pH of the mixture is adjusted with 50% NaOH to the pH indicated in Table 5 under stirring followed by shaking for another 15 minutes. The neutralized composition contains the dry solids content, glycerol content and water content as indicated in Table 5. The resulting aqueous solutions are prepared and tested to determine the flowback numbers in accordance with the method described in Example 5. The results of the testing are set forth below in Table 5.

TABLE 5
Flowback Number of Formulated Samples with Pour Point Depressant and
no added Free Fatty Acid

| | Crude | Dry solid | | | | |
|---|---|---|---|---|---|---|
| Formulated | Sophorolipid | content | | Neutralized | | 2% KCl |
| Sample | Sample | excluding | USP Glycerol | composition | Water | Flowback |
| (No.) | (No.) | glycerol (%) | (%) | Final pH | (%) | Number |
| 6-1 | 2-2 | 23 | 52 | 7.1 | 25 | 80 |
| 6-2 | 2-3 | 22 | 53 | 7.2 | 25 | 83 |
| 6-3 | 2-3 | 5 | 70 | 7.4 | 25 | 63 |
| 6-4 | 2-3 | 10 | 65 | 7.1 | 25 | 76 |
| 6-5 | 2-4 | 23 | 52 | 7.0 | 25 | 85 |
| 6-6 | 2-5 | 10 | 60 | 7.1 | 25 | 76 |
| 6-7 | 2-6 | 10 | 65 | 7.5 | 25 | 69 |

### Example 7

### Flowback Number with Pour Point Depressant and Added Free Fatty Acid

Crude sophorolipid of samples 2-3, 2-4 and 2-5 are mixed with the glycerol, fatty acid distillate, and Ultra-Pure Water in a sample tube to enable the creation of formulated samples as indicated in Table 6 (and further described below). The sample tube is placed on a shaker for 5 minutes and the pH of the mixture is adjusted with 50% NaOH to the pH indicated in Table 6 under stirring followed by shaking for another 15 minutes. The resulting aqueous solutions are prepared and tested to determine the flowback numbers in accordance with the method described in Example 5. The results of the testing are set forth below in Table 6.

TABLE 6
Flowback Number of Formulated Samples with Pour Point Depressant and
added Free Fatty Acid.

| | | Dry solid | | | | | | |
|---|---|---|---|---|---|---|---|---|
| | | content %, | Formulated | | | | | Hard |
| | Crude | excluding | Free Fatty | | | | 2% KCl | Water |
| Formulated | Sophorolipid | glycerol | Acid | | | | Flowback | Flowback |
| Sample | Sample | and free | Content | *Glycerol | Water | | Number | Number |
| (No.) | (No.) | fatty acid | (%) | (%) | (%) | pH | (%) | (%) |
| 7-1 | 2-3 | 4.0 | 1.5 | 70.0 | 25.0 | 7.0 | 65 | — |
| 7-2 | 2-3 | 9.0 | 1.0 | 65.0 | 25.0 | 7.0 | 84 | — |
| 7-3 | 2-3 | 13.5 | 2 | 60.0 | 25.0 | 7.4 | 86 | — |
| 7-4 | 2-4 | 4.5 | 0.6 | 70.0 | 25.0 | 8.0 | 70 | 66 |
| 7-5 | 2-4 | 3.6 | 0.5 | 71.0 | 25.0 | 8.8 | 68 | 57 |
| 7-6 | 2-4 | 17.4 | 0.8 | 57.03 | 25.0 | 8.4 | — | 75 |
| 7-7 | 2-4 | 16.9 | 1.3 | 57.02 | 25.0 | 8.7 | 88 | 78 |
| 7-8 | 2-5 | 17.5 | 0.8 | 56.9 | 25.0 | 8.3 | 89 | 77 |
| 7-9 | 2-5 | 17.0 | 1.3 | 56.9 | 25.0 | 8.0 | — | 75 |
| 7-10 | 2-5 | 4.5 | 0.6 | 70.0 | 25.0 | 7.6 | 67 | 63 |
| 7-11 | 2-5 | 3.6 | 0.5 | 71.0 | 25.0 | 8.8 | 66 | 58 |

*USP Glycerol is used for Samples 7-1 to 7-3; Crude Glycerol is used for Samples 7-4 to 7-11

### Example 8

### Pourability of Formulated Sophorolipid Samples with Pour Point Depressant and No Added Free Fatty Acid

Crude sophorolipid of samples 2-2 and 2-3 are mixed with the glycerol as indicated in Table 7 and Ultra-Pure Water in a sample tube. The sample tube is placed on a shaker for 5 minutes and the pH of the mixture is adjusted with 50% NaOH to the pH indicated in Table 7 under stirring followed by shaking for another 15 minutes. The sample is tested to determine its pourability in accordance with the procedure described in the analytical methods. The results of the testing are set forth below in Table 7. Formulated sophorolipid samples exhibit pourability at −20° C. (i.e., “flows”) for a minimum of 24 hours, all the samples exhibit pourability up to 8 weeks, sample 8-4 is pourable for 12 weeks, and sample 8-5 is pourable for 13 weeks.

TABLE 7
Pourability of Formulated Sophorolipid Samples with Pour Point Depressant
and no added Free Fatty Acid at −20° C.

| | Formulated Sample (No.) | | | | |
|---|---|---|---|---|---|
| | 8-1 | 8-2 | 8-3 | 8-4 | 8-5 |
| | Crude Sophorolipid Sample (No.) | | | | |
| | 2-2 | 2-2 | 2-2 | 2-3 | 2-3 |
| Dry solid content %, | 22.8 | 18.8 | 20.8 | 22.7 | 22.7 |
| excluding glycerol | | | | | |
| and free fatty acid | | | | | |
| *Glycerol (%) | 51.5 | 55.5 | 53.5 | 51.4 | 51.4 |
| Water (%) | 25.7 | 25.7 | 25.7 | 25.9 | 25.9 |
| pH | 7.1 | 7.1 | 7.1 | 6.8 | 7.0 |
| 24 hrs | Flows | Flows | Flows | Flows | Flows |
| Week 1 | Flows | Flows | Flows | Flows | Flows |
| Week 2 | Flows | Flows | Flows | Flows | Flows |
| Week 3 | Flows | Flows | Flows | Flows | Flows |
| Week 4 | Flows | Flows | Flows | Flows | Flows |
| Week 5 | Flows | Flows | Flows | Flows | Flows |
| Week 6 | Flows | Flows | Flows | Flows | Flows |
| Week 7 | Flows | Flows | Flows | Flows | Flows |
| Week 8 | Flows | Flows | Flows | Flows | Flows |
| Week 9 | — | — | — | Flows | Flows |
| Week 10 | — | — | — | Flows | Flows |
| Week 11 | — | — | — | Flows | Flows |
| Week 12 | — | — | — | Flows | Flows |
| Week 13 | — | — | — | Frozen | Flows |

*USP Glycerol is used for Samples 8-1, 8-2, 8-3, 8-5; Crude Glycerol is used for Sample 8-4
**Formulated samples 8-1, 8-2, and 8-3 were not evaluated past 8 weeks.

### Example 9

### Pourability of Formulated Sophorolipid Samples with Pour Point Depressant and with Added Free Fatty Acid

Crude sophorolipid of samples 2-4 and 2-5 are mixed with the glycerol and fatty acid distillate and Ultra-Pure Water in a sample tube to enable the creation of Formulated Samples as indicated in Table 8 (and as further described below). The sample tube is placed on a shaker for 5 minutes and the pH of the mixture is adjusted with 50% NaOH to the pH indicated in Table 8 under stiffing followed by shaking for another 15 minutes. The sample is tested to determine its pourability in accordance with the procedure described in the analytical methods. The results of the testing are set forth below in Table 8. Formulated sophorolipid samples exhibit pourability at −20° C. (i.e. “flows”) for a minimum of 24 hours.

TABLE 8
Pourability of Formulated Sophorolipid Samples with our Point Depressant
and added Free Fatty Acid at −20° C.

| | | Dry solid | | | | | |
|---|---|---|---|---|---|---|---|
| | | content %, | Formulated | | | | |
| | Crude | excluding | Free Fatty | | | | |
| Formulated | Sophorolipid | glycerol | Acid | | | | |
| Sample | Sample | and free | Content | Crude Glycerol | Water | | Pourability |
| (No.) | (No.) | fatty acid | (%) | (%) | (%) | pH | at 24 hrs |
| 9-1 | 2-4 | 4.5 | 0.6 | 70.0 | 25.0 | 8.0 | Flows |
| 9-2 | 2-4 | 3.6 | 0.5 | 71.6 | 25.0 | 8.8 | Flows |
| 9-3 | 2-4 | 18.2 | 0.8 | 57.0 | 25.0 | 8.4 | Flows |
| 9-4 | 2-4 | 18.2 | 1.3 | 57.0 | 25.0 | 8.7 | Flows |
| 9-5 | 2-5 | 18.2 | 0.8 | 56.9 | 25.0 | 8.3 | Flows |
| 9-6 | 2-5 | 18.2 | 1.3 | 56.9 | 25.0 | 8.0 | Flows |
| 9-7 | 2-5 | 4.5 | 0.6 | 70.0 | 25.0 | 7.6 | Flows |
| 9-8 | 2-5 | 3.6 | 0.5 | 71.0 | 25.0 | 8.8 | Flows |

### Example 10

### Flowback Number with No Added Pour Point Depressant and with Added Free Fatty Acid

Crude sophorolipid of samples 2-4 and 2-5 are mixed with fatty acid distillate and Ultra-Pure Water in a sample tube to enable the creation of Formulated Samples as indicated in Table 9. The sample tube is placed on a shaker for 5 minutes and the pH of the mixture is adjusted with 50% NaOH to the pH indicated in Table 6 under stirring followed by shaking for another 15 minutes. The resulting aqueous solutions are prepared and tested to determine the flowback numbers in accordance with the method described in Example 5. The results of the testing are set forth below in Table 9.

TABLE 9
Flowback Number of Formulated Samples with no added Pour Point
Depressant and with added Free Fatty Acid.

| | | Dry solid | | | | | |
|---|---|---|---|---|---|---|---|
| | | content (%), | Formulated | | | | |
| | Crude | excluding | Free Fatty | | | | |
| Formulate | Sophorolipid | glycerol | Acid | | | 2% KCl | Hard Water |
| Sample | Sample | and free | Content | Water | | Flowback | Flowback |
| (No.) | (No.) | fatty acid | (%) | (%) | pH | Number | Number |
| 10-1 | 2-4 | 8.9 | 1.1 | 89.9 | 7.1 | 83 | 61 |
| 10-2 | 2-4 | 4.5 | 0.6 | 94.9 | 8.1 | 70 | 61 |
| 10-3 | 2-4 | 3.6 | 0.5 | 95.9 | 8.1 | 70 | 61 |
| 10-4 | 2-5 | 8.9 | 1.1 | 89.9 | 7.2 | 84 | 62 |
| 10-5 | 2-5 | 8.5 | 1.6 | 89.9 | 7.1 | 77 | — |
| 10-6 | 2-5 | 4.5 | 0.6 | 94.9 | 7.3 | 70 | 59 |
| 10-7 | 2-5 | 3.6 | 0.5 | 96.0 | 7.2 | 67 | 61 |

1. A process to produce a sophorolipid composition, the process comprising:
(a) obtaining a sophorolipid containing composition comprising about 4-80 percent by weight total dry solids, comprising at least one sophorolipid, wherein the sophorolipid containing solution exhibits a pH of less than 5,
(b) adding about 6 percent by weight or less of a least one free fatty acid to the sophorolipid containing composition to provide a fatty acid adjusted sophorolipid composition, and
(c) adjusting the pH of the fatty acid adjusted sophorolipid composition to a pH greater than 5 to provide the sophorolipid composition.

2. The process of claim 1, wherein the total dry solids of (a) comprises from about 40 to about 99 percent by weight total sophorolipids based on the total dry solids.

3-9. (canceled)

10. The process of claim 13, wherein the sophorolipid containing composition of (a) comprises less than 75 percent by weight water.

11-12. (canceled)

13. The process of claim 1, wherein the sophorolipid containing composition of (a) comprises 25-96 percent by weight water.

13. (canceled)

14. The process of claim 1, wherein separating the sophorolipid layer from an aqueous layer comprises heating a fermentation broth containing sophorolipid to a temperature of about 70-75° C. to provide a heated broth, cooling the heated broth to an ambient temperature, and decanting a higher density sophorolipid layer from a lower density aqueous layer.

15. The process of claim 1, wherein the at least one sophorolipid comprises ester-form sophorolipid and acidic-form sophorolipid.

16. The process of claim 15, wherein a ratio of ester-form sophorolipid to acidic-form sophorolipid is at least 1:1.

17-18. (canceled)

19. The process of claim 16, wherein a ratio of ester form sophorolipid to acidic-form sophorolipid is not greater than 9:1.

20. The process of claim 19, wherein the fatty acid adjusted sophorolipid composition exhibits a pH of 12.5 or less.

21. (canceled)

22. The process of claim 1, wherein the fatty acid adjusted sophorolipid composition exhibits a pH of from about 6 to about 9.5.

23. The process of claim 1, wherein sufficient free fatty acid is added to obtain an adjusted sophorolipid composition having a formulated free fatty acid content from about 0.1 to about 6 percent by weight of the fatty acid adjusted sophorolipid composition.

24. (canceled)

25. The process of claim 1, wherein f sufficient free fatty acid is added to obtain an adjusted sophorolipid composition having a formulated free fatty acid content from about 0.5 to about 2.5 percent by weight of the fatty acid adjusted sophorolipid composition.

26-37. (canceled)

38. The process of claim 1, wherein the added free fatty acid comprises oleic acid.

39. The process of claim 1, wherein oleic acid comprises a majority of the added free fatty acid.

40. The process of claim 1, wherein the added free fatty acid comprises a free fatty acid, a neutralized fatty acid salt, a free fatty acid anion distributed in an aqueous sophorolipid containing composition, or mixtures thereof.

41. The process of claim 1, wherein the added free fatty acid is obtained from a fatty acid distillate derived from plant-based oil, animal fat or fish oil.

42. The process of claim 1 further comprising adding a biocide to the fatty acid adjusted sophorolipid composition.

43. The process of claim 1 further comprising adding a biocide effective at a pH of about 5-12.

44. The process of claim 42, wherein amounts of effective biocides are about at least 10 ppm (0.001 percent by weight).

45. The process of claim 24, wherein amounts of effective biocides are about less than 1 percent by weight (10000 ppm), less than 0.5 percent by weight (5000), or less than 0.05 percent by weight (500 ppm).

46-90. (canceled)
